(19) United States
(12) Patent Application Publication
(10) Pub. No.: US 2010/0055041 A1
(43) Pub. Date: Mar. 4, 2010

(54) NANOTRACER FOR IN-SITU GASTRIC CANCER DETECTION

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/200,566

(22) Filed: Aug. 28, 2008

(51) Int. Cl. A61K 49/00

(52) U.S. Cl. 424/9.35; 424/9.1; 424/9.6; 424/9.3; 424/9.4; 977/905

(57) ABSTRACT

Nanotracers for in-situ detection of cancerous gastric tissue include substantially monodisperse polymeric nanoparticles, at least one detectable label incorporated into each particle, and receptor-specific ligands coupled to each nanoparticle. The receptor-specific ligand is selected such that it interacts preferentially with ligand-specific receptors that are overexpressed on cancerous cells.

### BACKGROUND

Gastric cancer is the fourth most common cancer worldwide with about 1 million cases diagnosed in a given year. Gastric cancer causes about 700,000 deaths per year making it the second most common cause of cancer death worldwide after lung cancer. In the United States, gastric cancer represents roughly 2% of all cancers diagnosed per year. Gastric cancer is much more common in countries such as Korea, Japan, Great Britain, South America, and Iceland.

Infection with the bacterium Helicobacter pylori is the main risk factor in about 80% or more of gastric cancers. Additional risk factors include a high salt diet, smoking, and low intake of fruits and vegetables. Gastric cancer is more common in men, with about 3 cases being diagnosed in men for every 1 diagnosed in women.

Gastric cancer can develop in any part of the stomach and may spread throughout the stomach and to other organs; particularly the esophagus and the small intestine. Metastasis occurs in 80-90% of individuals with gastric cancer, with a five year survival rate of 75% in those diagnosed in early stages and less than 30% of those diagnosed in late stages.

Gastric cancer may be suspected based on a set of non-specific symptoms such as chronic indigestion or heartburn, loss of appetite, especially for meat, abdominal pain or discomfort in the upper abdomen, nausea, bleeding, and weight loss among others. If gastric cancer is suspected, a doctor may order other tests such as an endoscopic exam and/or a tissue biopsy.

Surgery is the most common treatment for gastric cancer. The surgeon removes part or all of the stomach, as well as some of the tissue around the stomach, with the basic goal of removing all cancer and a margin of normal tissue. Depending on the extent of invasion and the location of the tumor, surgery may also include removal of part of the intestine or pancreas. Surgical interventions are currently curative in less than 40% of cases, and, in cases of metastasis, may only be palliative.

However, identifying the extent of the gastric cancers can be difficult. It is important for the physician to remove all of the cancerous tissue, but at the same time it is undesirable in terms of the patient's quality of life after surgery for the physician to remove excess healthy tissue. Gastric cancers are notorious for having no consistent biomarkers that can allow easy identification of cancerous regions. As a result, decisions about how much of the stomach to remove are often made after the physician has opened the abdomen and can visually inspect the stomach.

### BRIEF SUMMARY

The illustrated embodiments relate to novel nanotracers for in-situ detection of cancerous gastric tissue and methods for detecting gastric cancer and differentiating healthy tissue from cancerous tissue. The nanotracers are prepared by forming nanoparticles that incorporate a detectable label. A receptor-specific ligand, which is capable of interacting with cellular receptors, is coupled to the nanoparticles. In-situ detection of cancerous gastric tissue can, for example, assist surgeons in deciding how much of the gastric organ should be removed as part of surgical treatment.

In one embodiment, a nanotracer for in-situ detection of cancerous gastric tissue is disclosed. In one embodiment, the nanotracer includes a nanoparticle, a detectable label associated with the nanoparticle, and a receptor-specific ligand coupled to the nanoparticle.

In another embodiment, a method for in-situ detection of cancerous gastric tissue is disclosed. The method includes (1) exposing the gastric tissue to nanotracers, where each nanotracer includes a nanoparticle, a detectable label disposed in or associated with the nanoparticle, and a receptor-specific ligand coupled to the nanoparticle, and (2) allowing the receptor-specific ligand to associate with the gastric tissue, wherein the nanotracer associates with the cancerous gastric tissue via the receptor-specific ligand.

In one embodiment, the receptor-specific ligand molecules are configured such that they associate preferentially with ligand specific receptors displayed on cells of the cancerous gastric tissue. In one embodiment, the method further includes a step of visualizing the cancerous gastric tissue by detecting the nanotracer via the detectable label.

In one embodiment, the nanoparticles are substantially monodisperse. In one embodiment, the nanoparticles have a size in a range of about 1 nm to about 2000 nm. In another embodiment, the nanoparticles have a size in a range of about 50 nm to about 1500 nm. In yet another embodiment, the nanoparticles have a size in a range of about 100 nm to about 1000 nm.

In one embodiment the nanoparticles are composed of a polymer. Suitable examples of polymers include, but are not limited to, homopolymers or copolymers of polystyrene, poly(methyl methacrylate), polyacrylamide, poly(ethylene glycol), poly(hydroxyethylmethacrylate), poly(vinyltoluene), or poly(divinylbenzene), and combinations thereof.

In one embodiment, the nanoparticle includes a detectable label selected from the group consisting of a metallic particle, a fluorescent dye, a quantum dot, a quantum barcode, a radiographic contrast agent, or a magnetic resonance imaging contrast agent, and combinations thereof.

The detectable label facilitates detection of the cancerous tissue and differentiation of cancerous tissue from healthy tissue. The detectable label can be detected using any one of a number medical imaging technologies including, but not limited to, x-ray, CT scan, x-ray fluoroscopy, fluorescence, or magnetic resonance imaging, and combinations thereof.

In one embodiment, each nanotracer displays functional groups for attachment of receptor specific ligand molecules thereto. Suitable example of functional groups include, but are not limited to, one or more members selected from the group of a hydroxyl, a carboxyl, a carbonyl, an amine, an amide, a nitrile, a nitrogen with a free lone pair of electrons, an amino acid, a thiol, imidazole, phosphonic acid, phosphinic acid, a sulfonic acid, a sulfonyl halide, or an acyl halide.

In one embodiment, the receptor-specific ligand that is attached to the nanotracer includes a nutritive substance selected from the group consisting of sugars, vitamins, amino acids, or DNA bases, and combinations thereof.

In one embodiment, suitable examples of nutritive substances include, but are not limited to, at least one water-partitionable vitamin is selected from the group consisting of vitamin B1, vitamin B2, vitamin B3, vitamin B5, vitamin B6, vitamin B7, vitamin B9, vitamin B12, or vitamin C, and combinations thereof.

In one embodiment, suitable examples of nutritive substances include, but are not limited to, at least one lipid-partitionable vitamin chosen from the group consisting of vitamin A, vitamin D, vitamin E, or vitamin K, and combinations thereof.

In one embodiment, the nutritive substance is folic acid or folate.

These and other objects and features of nanotracers for in-situ detection of gastric cancer will become more fully apparent from the following description and appended claims, or may be learned by the practice of the claims as set forth hereinafter.

The foregoing summary is illustrative only and is not intended to be in any way limiting. In addition to the illustrative aspects, embodiments, and features described above, further aspects, embodiments, and features will become apparent by reference to the following detailed description.

### BRIEF DESCRIPTION OF THE DRAWINGS

To further clarify the above and other advantages and features of nanotracers for in-situ detection of gastric cancer, a more particular description will be rendered by reference to specific embodiments thereof which are illustrated in the appended drawings. It is appreciated that these drawings depict only typical embodiments of nanotracers for in-situ detection of gastric cancer and are therefore not to be considered limiting of in scope. Nanotracers for in-situ detection of gastric cancer will be described and explained with additional specificity and detail through the use of the accompanying drawings in which:

FIG. 1A illustrates a schematic view of a nanotracer having one type receptor specific ligands associated therewith;

FIG. 1B illustrates a schematic view of a nanotracer having multiple types of receptor specific ligands associated therewith;

FIG. 2A illustrates a schematic view of the inside of a stomach with a cancerous region;

FIG. 2B another view of the stomach depicted in FIG. 2A in which the gastric cancer is associated with nanotracers.

### DETAILED DESCRIPTION

The illustrative embodiments described in the detailed description and claims are not meant to be limiting. Other embodiments may be utilized, and other changes may be made, without departing from the spirit or scope of the subject matter presented here.

### I. Introduction

The illustrated embodiments relate to novel nanotracers for in-situ detection of cancerous gastric tissue and methods for detecting gastric cancer and differentiating healthy tissue from cancerous tissue. The nanotracers are prepared by forming nanoparticles that incorporate a detectable label. A receptor-specific ligand, which is capable of interacting with cellular receptors, is coupled to the nanoparticles.

Cancerous tissues generally divide more rapidly than healthy tissues, and have, as a result, greater nutritive needs than their healthy counterparts. In addition, cancerous tissues produce disproportionate numbers of ligand receptors that are responsible for binding and transporting certain nutrients into the cell. In some embodiments, nanotracers allowing for in-situ detection of gastric cancers associate with the cancerous tissue via receptor specific ligands, and detection of the associated detectable label permits the differentiation of healthy gastric tissue from cancerous gastric tissue. In-situ detection of cancerous gastric tissue can, for example, assist surgeons in deciding how much of the gastric organ should be removed as part of surgical treatment.

As used herein, the term “cancerous” refers to diseased gastric cells including, but not limited to, pre-cancerous cells, cancerous cells, neoplastic cells, and cells displaying morphological dysplasia.

As used herein, the terms “gastric cancer” or “stomach cancer” refer to cancers of the stomach. The most common types of gastric cancer are carcinomas, such as but not limited to, adenocarcinomas, affecting the epithelial cells of the stomach. Stomach cancers may additionally include, for example, sarcomas affecting the connective tissue of the stomach and blastomas affecting the blast tissue of the stomach.

As used herein, the terms “nano-scale,” “nano-sized,” or “nanoparticle” refer to particles having a size between 1 nm and 2000 nm.

As used herein, the term “monodisperse” refers to a population of objects having a relatively narrow size distribution.

### II. Nanotracers

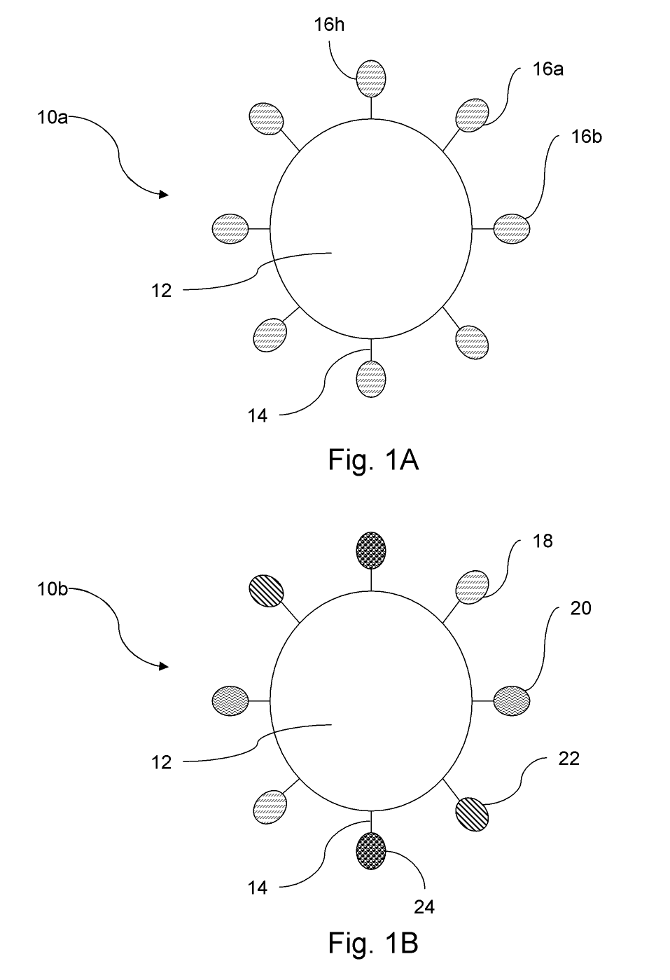

In one embodiment, a nanotracer for in-situ detection of cancerous gastric tissue is disclosed. Illustrative nanotracers are schematically represented in FIGS. 1A and 1B.

In one embodiment depicted in FIG. 1A, a nanotracer 10a includes a nanoparticle that includes a detectable label 12, and receptor-specific ligands 16a-16h operably coupled to the nanoparticle 12. The receptor-specific ligands 16a-16h are operably coupled to the nanoparticle 12 through functional groups 14 on the outer surface of the nanoparticle 12. Receptor specific ligands 16a-16h are able to associate with ligand-specific receptors found on the cells of the stomach.

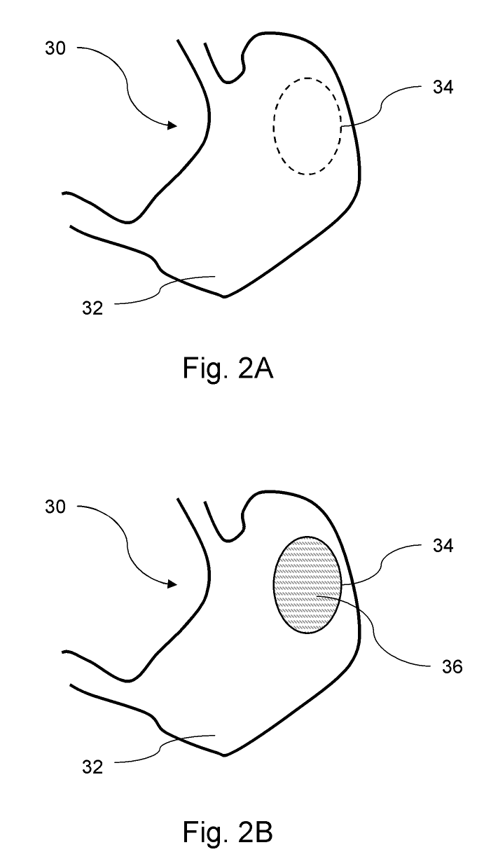

In the embodiment depicted in FIG. 1A, the nanotracer 10a is associated with one type of receptor specific ligand 16a-16h. FIG. 1B depicts another embodiment of a nanotracer 10b. Nanotracer 10b includes a nanoparticle that includes a detectable label 12, and multiple types of receptor-specific ligands (18, 20, 22, and 24) operably coupled to the nanoparticle 12. In FIG. 2B, different types of receptor-specific ligands are schematically depicted by the different textures shown in the ligand molecules 18, 20, 22, and 24. In some embodiments, it may be advantageous to use nanotracers having multiple types of receptor-specific ligands. For example, it may be advantageous if it is unknown what type of cancer the subject is afflicted with. And while nanotracer 10b is shown having four types of receptor specific ligands 18, 20, 22, and 24, more or fewer types may be included in other embodiments (not shown).

a. Nanoparticles

According to the illustrated embodiments, the nanotracer includes nanoparticles that are substantially monodisperse, meaning that they are prepared such that a given population has a relatively narrow size distribution. Having a narrow size range can allow the nanoparticles to interact with certain tissue types preferentially. For example, clusters of cancerous cells tend to have larger interstitial spaces between cells relative to normal, healthy tissues. As a result, larger particles within a narrow size range may tend to interact preferentially with cancerous tissue. Moreover, using particles having a narrow size range produces a more uniform signal from the detectable label.

In one embodiment, the nanoparticles have a size in a range of about 1 nm to about 2000 nm. In another embodiment, the nanoparticles have a size in a range of about 50 nm to about 1500 nm. In yet another one embodiment, the nanoparticles have a size in a range of about 100 nm to about 1000 nm. In some embodiments, the nanparticles have a size in a range from about 1 nm, 5 nm, 10 nm, 50 nm, 100 nm, 250 nm, 500 nm, 750 nm, 1000 nm, or 1500 nm, to about 5 nm, 10 nm, 50 nm, 100 nm, 250 nm, 500 nm, 750 nm, 1000 nm, 1500 nm, or 2000 nm. In some embodiments, the nanoparticles have a size of about 1 nm, 5 nm, 10 nm, 50 nm, 100 nm, 250 nm, 500 nm, 750 nm, 1000 nm, 1500 nm, or 2000 nm.

In one embodiment, the nanotracer is formed by polymerizing a monomer material to form polymeric nanoparticle. For example, biologically compatible polymeric nanospheres can be formed by polymerization of monomer materials in aqueous solution with or without the inclusion of surfactants and/or crosslinking agents. One or more detectable labels may be incorporated into the nanoparticle either during or after polymerization of the monomer material.

In a broad embodiment, suitable examples of polymeric material include, but are not limited to, acrylic acid, or any ester thereof, such as methyl acrylate, ethyl acrylate, propyl acrylate, butyl acrylate, 2-ethyl hexyl acrylate or glycidyl acrylate, methacrylic acid, or any ester thereof, such as methyl methacrylate, ethyl methacrylate, propyl methacrylate, butyl methacrylate, lauryl methacrylate, cetyl methacrylate, stearyl methacrylate, ethylene glycol dimethacrylate, tetraethylene glycol dimethacrylate, glycidyl methacrylate or N,N-(methacryloxy hydroxy propyl)-(hydroxy alkyl)amino ethyl amidazolidinone, allyl esters such as allyl methacrylate, itaconic acid, or esters thereof, crotonic acid, or esters thereof, maleic acid, or esters thereof, such as dibutyl maleate, dioctyl maleate, dioctyl maleate or diethyl maleate, styrene, or substituted derivatives thereof such as ethyl styrene, butyl styrene or divinyl benzene, monomer units which include an amine functionality, such as dimethyl amino ethyl methacrylate or butyl amino ethyl methacrylate, monomer units which include an amide functionality, such as acrylamide or methacrylamide, vinyl-containing monomers such as vinyl ethers; vinyl thioethers; vinyl alcohols; vinyl ketones; vinyl halides, such as vinyl chlorides; vinyl esters, such as vinyl acetate or vinyl versatate; vinyl nitriles, such as acrylonitrile or methacrylonitrile, vinylidene halides, such as vinylidene chloride and vinylidene fluoride, tetrafluoroethylene, diene monomers, such as butadiene and isoprene, and allyl ethers, such as allyl glycidyl ether.

In a narrower embodiment, suitable example of polymeric materials include, but are not limited to, homopolymers or copolymers of polystyrene, poly(methyl methacrylate), polyacrylamide, poly(ethylene glycol), poly(hydroxyethylmethacrylate), poly(vinyltoluene), or poly(divinylbenzene), and combinations thereof.

b. Detectable Labels

In one embodiment, the nanoparticle includes a detectable label selected from the group consisting of a fluorescent dye, a quantum dot, a quantum barcode, a metallic particle, a radiographic contrast agent, or a magnetic resonance imaging contrast agent, and combinations thereof.

The detectable label facilitates detection of the cancerous tissue and differentiation of cancerous tissue from healthy tissue. The detectable label can be detected using any one of a number medical imaging technologies including, but not limited to, x-ray, CT scan, x-ray fluoroscopy, fluorescence, or magnetic resonance imaging, and combinations thereof.

In one embodiment, one or more fluorescent dyes are incorporated into the polymeric nanoparticles. Examples of suitable fluorescent dyes include, but are not limited to, fluoroscein, fluoroscein isothiocyanate, rhodamine dyes, coumarin dyes, luciferin, the AlexaFluor™ family of fluorescent dyes produced by Molecular Probes, or the DyLight Fluor™ family of fluorescent dyes is produced by Thermo Fisher Scientific.

Fluorescent dyes can be incorporated by a number of means either during or after formation of nanoparticles. U.S. Pat. Nos. 4,326,008, 4,267,235, 5,073,498, 5,952,131, 4,613,559, 5,395,688, 4,829,101, 4,996,265, 5,723,218, 5,786,219, 5,326,692, 5,573,909, 5,266,497, 4,613,559, 4,487,855, 5,194,300, 4,774,189, 3,790,492, 6,964,747, which are incorporated herein by specific reference in their entirety, discuss various methods for incorporating fluorescent dyes into polymeric particles. Typical methods include but are not limited to copolymerization, partitioning of water-soluble or oil-soluble dyes into particles by cosolublization of the dye and the monomer materials in various aqueous and non-aqueous solvents, attachment of the dye by functionalization of internal or external particle surfaces, and encapsulation of the dye by swelling the particle after forming and incorporation of the dye into the resulting spaces.

In one embodiment, a quantum dot or a quantum barcode is incorporated into the nanoparticle. Typically the quantum dot or barcode is first provided and subsequently coated with a polymeric material. Methods for manufacturing mondisperse quantum dots and quantum barcodes are known by persons having skill in the art. For example, quantum dots can be synthesized colloidally from precursor compounds dissolved in solutions based on a three component system composed of: precursors, organic surfactants, and solvents. Further discussion of colloidal synthesis of quantum dots can be found in “Colloidal synthesis of nanocrystals and nanocrystal superlattices,” IBM J. Res. & Dev. vol. 45, No. 1, January 2001, pp. 47-56 by C. B. Murray, which is incorporated herein by specific reference in its entirety.

A quantum dot typically consists of a semiconductor nanocrystal (e.g., CdSe) surrounded by a passivation shell (e.g., ZnS). Upon absorption of a photon, an electron-hole pair is generated, the recombination of which in ˜10-20 ns leads to the emission of a less-energetic photon. This energy, and therefore the wavelength, is dependent on the size of the quantum dot particle (smaller particles emit at a lower wavelength), which can be varied almost at will by controlled-synthesis conditions.

In a sense, a quantum dot functions in many ways like a fluorescent dye, except that they are more versatile in their excitation and emission wavelengths, their quantum yield is typically higher, and they are not subject to photobleaching in the way that fluorescent dyes typically are.

A quantum barcode has properties similar to a quantum dot except that it absorbs a broad spectrum of light and emits a specific pattern of wavelengths that acts as a particular signature or “barcode.” Typically, a quantum barcode is several different types of quantum dots, each having a particular emission spectrum, that are arranged in a multi layered shell or side-by-side fashion. Quantum barcodes are advantageous at least insofar as their emission pattern produces a particular signature that can be easily detected and tracked.

Metallic particles of a number of types can be incorporated into the nanoparticles either by providing metallic nanoparticles particles or preparing them in situ and coating them with one or more of the a polymer materials as discussed above. Suitable examples of metallic particles that are useful as detectable labels include, but are not limited to, ferric iron oxide (Fe₂O₃) and/or other ferric iron compounds, gadolinium metal or gadolinium-containing compounds, barium sulfate (BaSO₄), or nanogold particles, and combinations thereof.

In one embodiment, the nanoparticles include a detectable label that is a radiographic contrast agent. Radiographic contrast agent can, for example, allow for x-ray imaging of soft tissues such as gastric tissues. In the presently disclosed embodiments, the radiographic contrast agent is included to permit medical personnel to distinguish between cancerous and healthy gastric tissue. Suitable examples of radiographic contrast agents that can be incorporated into nanoparticles include, but are not limited to, barium sulfate (BaSO₄) nanoparticles, nanogold particles, iodine-based x-ray contrast agents, and other materials that include heavy nuclei that efficiently absorb x-rays.

In one embodiment, the nanoparticles include a detectable label that is a nuclear magnetic resonance imaging (MRI) contrast agent. While MRI is typically quite useful for imaging soft tissues, the use of contrast agents is common when imaging the GI tract because it can be difficult to distinguish between the GI tract and the other abdominal organs. In the presently disclosed embodiments, MRI contrast agents are included to permit medical personnel to distinguish between cancerous and healthy gastric tissue. Suitable examples of MRI contrast agents include, but are not limited to, ferric iron oxide (Fe₂O₃) and/or other ferric iron compounds, gadolinium metal or gadolinium-containing compounds, materials containing protons in —CH₂— groups, and compounds containing MRI active nuclei that are not naturally abundant in the body, such as helium-3, carbon-13, fluorine-19, oxygen-17, sodium-23, phosphorus-31, and xenon-129.

Ferric iron and gadolinium compounds are paramagnetic agents that shorten the proton spin relaxation times in surrounding water molecules. Materials containing protons in —CH₂— groups relax at a faster rate than in water resulting in detectable change in the MRI signal. In the presently disclosed embodiments, the polymeric materials that make up the nanoparticles are rich in protons in —CH₂— groups, which means that the nanoparticles of the nanotracer can act as an MRI contrast agent.

c. Receptor-Specific Ligands

In one embodiment, molecules of a receptor-specific ligand are coupled to the nanotracer. In one embodiment, each nanotracer includes functional groups for attachment of receptor-specific ligands thereto. That is, the polymeric material may contain functional groups that provide sites for the attachment of receptor-specific ligands desirable for binding to ligand receptors on the cancerous tissue. Suitable examples of functional groups include, but are not limited to, one or more members selected from the group of a hydroxyl, a carboxyl, a carbonyl, an amine, an amide, a nitrile, a nitrogen with a free lone pair of electrons, an amino acid, a thiol, imidazole, phosphonic acid, phosphinic acid, a sulfonic acid, a sulfonyl halide, or an acyl halide.

In one embodiment, the receptor-specific ligand that is coupled to the nanoparticle is a nutritive substance. Nutritive substances facilitate the identification of cancerous gastric tissue because cancerous cells typically have greater nutritive needs. Although not intending to be limited by a particular theory, it is generally believed that cancerous tissues have greater nutritive needs because they are growing and dividing more rapidly than “normal” cells. Apparently as a result, cancerous cells typically express disproportionate numbers of receptors for nutritive ligands.

Suitable examples of nutritive substances include, but are not limited to, sugars, vitamins, amino acids, or DNA bases, and combinations thereof. For example, the nutritive substance may be at least one water-partitionable vitamin selected from the group consisting of vitamin B1, vitamin B2, vitamin B3, vitamin B5, vitamin B6, vitamin B7, vitamin B9, vitamin B12, or vitamin C, and combinations thereof. In another example, the nutritive substance may be at least one lipid-partitionable vitamin chosen from the group consisting of vitamin A, vitamin D, vitamin E, or vitamin K, and combinations thereof.

In an illustrative embodiment, the nutritive substance is folic acid or folate. Folic acid and folate ion are also known as vitamin B₉. It has been found that attaching folate to the functional groups on the nanoparticle via the y-carboxylate group allows folate to bind normally to folate receptors. Additional discussion of folate receptor binding can be found in Leamon, C. P. and Low, P. S. “Delivery of Macromolecules Into Living Cells: A Method That Exploits Folate Receptor Endocytosis” (1991) Cell Biology, 88:5572-5576, which is incorporated herein by reference in its entirety.

Folic acid or folate are needed to synthesize DNA bases (most notably thymine, but also purine bases) needed for DNA replication. As a result, folic acid or folate deficiency hinders DNA synthesis and cell division. While all cells need folic acid or folate to a certain extent, the need is most keen in rapidly dividing cells such as cancerous cells.

Because they need more folic acid or folate than normal cells, cancerous cells of many types have been found to express disproportionate numbers of folate receptors. The presence of elevated receptor populations can correlate with the aggressive or proliferative status of tumor cells. Folate receptors have also been shown to recycle during the internalization of folate in rapidly dividing cells such as cancer cells. Thus, folate and folate receptors are considered to be useful as tumor markers for gastric cancer and are targets for nanotracers intended to identify cancerous cells.

II. Methods for in-situ Detection of Cancerous Gastric Tissue

In one embodiment, a method for in-situ detection of cancerous gastric tissue is disclosed. The method includes exposing the gastric tissue to nanotracers, where each nanotracer includes a nanoparticle, a detectable label disposed in or associated with the nanoparticle, and a receptor-specific ligand coupled to the nanoparticle; and allowing the receptor-specific ligand to associate with the gastric tissue, wherein the nanotracer associates with the cancerous gastric tissue via the receptor-specific ligand.

In one embodiment, the gastric tissue can be exposed to the nanotracers by having the subject swallow a composition, such as, bur not limited to, a palatable shake-like composition that well known to those in the art. In another embodiment, nanotracers can be introduced directly into the stomach by means such as, but not limited to, a nasogastric feeding tube or an endoscopic tube. In yet another embodiment, nanotracers can be applied directly to the gastric tissue during a surgical procedure. The amount of nanotracers that the gastric tissue is dependent at least in part on the type of cancer, the type or types of receptor-specific ligands used, and the method of detection.

When a subject's gastric tissue is exposed to nanotracers, the receptor specific ligands associate with the receptors that are displayed on the gastric cells. While the nanotracers are capable of binding to all cells, it has been found that cancerous cells frequently overexpress certain receptors, including, for example, the receptor for folic acid. Because cancerous cells overexpress receptor molecules, the nanotracers will bind preferentially to the cancerous cells. Such preferential binding can allow differentiation between masses of cancerous cells and healthy cells. For example, even if cancerous cells only express 10% more receptors than healthy cells, modern image and data processing techniques can allow healthy tissue to be distinguished from cancerous tissue.

FIGS. 2A and 2B schematically depict a stomach 30 before and after being exposed to nanotracers. In FIG. 2A, the interior of the stomach 32 includes a cancerous region 34 that is indicated by the region bounded by the dotted line. However, it is often difficult to determine the extent of a gastric cancer by visual inspection. As a result, surgeons often err on the side of caution and remove more gastric tissue than is necessary to excise the cancer. FIG. 2B depicts the same stomach as in FIG. 2A except the stomach has been exposed to nanotracers. The nanotracers preferentially bind in the cancerous region 36. The extent of the cancerous region 34 can be determined using the techniques described in detail below. Determining the extent of the cancerous region either before or during surgery allows surgeons and other medical personnel to excise the cancerous tissue without removing excess stomach tissue.

Nanotracers can be detected by a number of means, including, but not limited to, fluorescently, radiographically, and with nuclear magnetic resonance (MRI) imaging. Nanotracers can be detected fluorescently, radiographically, MRI imaging by virtue of the inclusion of fluorescently active, radiologically active, and MRI active detectable labels in the nanoparticles. A more detailed discussion of the various detectable labels can be found in the previous section.

Fluorescent detection can be used to detect nanotracers that include detectable labels such as fluorescent dyes, quantum dots, and quantum barcodes. Fluorescent detection may, for example, involve inserting an endoscopic camera that includes a light source capable of exciting the fluorescent label into the subject's stomach and collecting a series of images of the interior of the stomach that can be used to identify cancerous regions for later treatment.

Radiography is the use of x-rays to view structures inside the body. Typically, x-rays are used to visualize dense structures such as bone, but, with the use of contrast agents, soft tissues such as the stomach can readily be visualized. Typical radiographic techniques include x-ray photography, computed tomography (CT) scanning, and x-ray fluoroscopy. In x-ray photography, x-rays are passed through the subject and the pattern of absorbance and transmission is detected with a detector such as but not limited to photographic film, a scintillator, or a semiconductor diode array. CT scanning is similar to standard x-ray techniques except digital geometry processing is used to generate a three-dimensional image of the inside of an object from a large series of two-dimensional x-ray images taken around a single axis of rotation. X-ray fluoroscopy is commonly used by physicians to obtain real-time moving images of the internal structures of a patient through the use of a fluoroscope. As with the fluorescence techniques, radiographic images of the stomach that can be used to identify cancerous regions for later treatment.

MRI uses powerful magnets in combination with radio waves to visualize the structure and function of the body. In brief, the magnet is used to align the nuclear magnetization or “spin” of certain atoms. Once the spins are aligned, radiofrequency fields are used to switch the polarity of the spin; realignment or “relaxation” of the spin produces a rotating magnetic field detectable by the MRI scanner as a signal. The signal is converted into an image by scanning across the mass that is being examined and correlating the amount of signal to the amount of active nuclei present in a given region.

The most common MRI techniques use hydrogen atoms in water in the body, but other nuclei such as ³H, ¹³C, ¹⁷O, ¹⁹F, ²³Na, ³¹P, and ¹²⁹Xe also possess spin and can be observed by MRI. Particular pulse sequences can be used to observe one type of nuclei (e.g., ¹H or ¹³C) while not observing other active nuclei that may be present.

Because water (i.e., ¹H) is present in essentially all soft tissues in the body, contrast agents or MRI active nuclei that are not naturally plentiful in the body are often used to enhance imaging of certain structures. As discussed in more detail above nanotracers having ferric iron oxide (Fe₂O₃) and/or other ferric iron compounds, gadolinium metal or gadolinium-containing compounds, materials containing protons in —CH₂— groups, and compounds containing MRI active nuclei such as helium-3, carbon-13, fluorine-19, oxygen-17, sodium-23, phosphorus-31, and xenon-129 can be used to enhance contrast of images of the stomach and differentiate between healthy and cancerous gastric tissue. Identification of the extent of cancerous tissue in the stomach can be used to guide later treatment.

In one embodiment, the receptor-specific ligand molecules are configured such that they associate preferentially with ligand specific receptors displayed on cells of the cancerous gastric tissue. For example, it has been found that cancerous cells overexpress ligand-specific receptors that are responsible for binding and transporting nutritive substances such as but not limited to sugars, amino acids, or folate. As a result, cancerous cells associate with greater numbers of nanotracers than healthy cells.

Nevertheless, the degree of preferential binding of nanotracers to cancerous cells may not allow for detection of cancerous tissue without use of data processing and image enhancement techniques. Data processing and image enhancement techniques can be used, for example, to enhance the contrast or subtract background in order to see features that cannot be seen in raw images. Data processing and image enhancement techniques can include, but are not limited to, contrast enhancement, baseline reduction, and image reconstruction using image and data processing, scalar field methods, vector field methods, tensor field methods, and diffusion tensor imaging.

Many commercial and open-source data processing packages are available for processing image data. For example, radiologists commonly use computer-aided diagnosis packages to analyze x-ray data and enhance features in medical x-rays. Suitable software packages for data processing, contrast enhancement, baseline reduction, image reconstruction, and feature identification include, but are not limited to, SCIRun/BioPSE/Uintah™, Fusion™, ITK™, and GemIdent™.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The nanotracers for in-situ detection of cancerous gastric tissue may be embodied in other specific forms without departing from the spirit or essential characteristics of this disclosure. The described embodiments are to be considered in all respects only as illustrative and not restrictive. All changes that come within the meaning and range of equivalency of the claims are to be embraced within their scope.

What is claimed is:

1. A nanotracer for in-situ detection of cancerous gastric tissue, the nanotracer comprising:
a nanoparticle that includes a detectable label; and
receptor-specific ligand molecules attached to the nanoparticle, wherein the receptor-specific ligand molecules are configured to associate with said cancerous gastric tissue.

2. A nanotracer as recited in claim 1, wherein the nanoparticles are substantially monodisperse.

3. A nanotracer as recited in claim 1, wherein the nanoparticle has a size in a range of about 1 nm to about 2000 nm.

4. A nanotracer as recited in claim 1, wherein the nanoparticle has a size in a range of about 50 nm to about 1500 nm.

5. A nanotracer as recited in claim 1, wherein the nanoparticle has a size in a range of about 100 nm to about 1000 nm.

6. A nanotracer as recited in claim 1, wherein the nanoparticle includes at least one polymer selected from the group consisting of homopolymers or copolymers of polystyrene, poly(methyl methacrylate), polyacrylamide, poly(ethylene glycol), poly(hydroxyethylmethacrylate), poly(vinyltoluene), or poly(divinylbenzene), and combinations thereof.

7. A nanotracer as recited in claim 1, wherein the nanoparticle includes a detectable label selected from the group consisting of a fluorescent dye, a quantum dot, a quantum barcode, a metallic particle, a radiographic contrast agent, or a magnetic resonance imaging contrast agent, and combinations thereof.

8. A nanotracer as recited in claim 1, wherein each nanotracer further includes functional groups for attachment of receptor-specific ligand thereto.

9. A nanotracer as recited in claim 1, wherein the receptor-specific ligand is a nutritive substance selected from the group consisting of sugars, vitamins, amino acids, or DNA bases, and combinations thereof.

10. A nanotracer as recited in claim 9, wherein the nutritive substance is at least one water-partitionable vitamin is selected from the group consisting of vitamin B₁, vitamin B₂, vitamin B₃, vitamin B₅, vitamin B₆, vitamin B₇, vitamin B₉, vitamin B₁₂, or vitamin C, and combinations thereof.

11. A nanotracer as recited in claim 9, wherein the nutritive substance is at least one lipid-partitionable vitamin is chosen from the group consisting of vitamin A, vitamin D, vitamin E, or vitamin K, and combinations thereof.

12. A nanotracer as recited in claim 9, wherein the nutritive substance is folic acid or folate.

13. A method for in-situ detection of cancerous gastric tissue, the method comprising:
exposing said gastric tissue to nanotracers, each nanotracer including:
a nanoparticle;
a detectable label disposed in or associated with the nanoparticle;
receptor-specific ligands coupled to the nanoparticle; and
wherein the nanotracer at least partially associates with said cancerous gastric tissue via the receptor-specific ligand.

14. A method as recited in claim 13, wherein the receptor-specific ligand molecules are configured such that they associate preferentially with ligand specific receptors displayed on cells of the cancerous gastric tissue.

15. A method as recited in claim 13, the method further including visualizing said cancerous gastric tissue by detecting the nanotracer via the detectable label.

16. A method as recited in claim 15, the method further including detecting the detectable label fluorescently.

17. A method as recited in claim 15, the method further including detecting the detectable label radiographically.

18. A method as recited in claim 15, the method further including detecting the detectable label using magnetic resonance imaging.

19. A method as recited in claim 13, wherein the nanoparticle includes a polymer selected from the group consisting of homopolymers or copolymers of polystyrene, poly(methyl methacrylate), polyacrylamide, poly(ethylene glycol), poly(hydroxyethylmethacrylate), poly(vinyltoluene), or poly(divinylbenzene), and combinations thereof.

20. A method as recited in claim 13, wherein the receptor-specific ligand is a nutritive substance selected from the group consisting of sugars, vitamins amino acids, or DNA bases, and combinations thereof.

21. A method as recited in claim 20, wherein the nutritive substance is folic acid or folate.

22. A nanotracer for in-situ detection of cancerous gastric tissue, the nanotracer comprising:
substantially monodisperse nanoparticles having a size in a range of about 1 nm to about 2000 nm, each nanoparticle including:
a polymer selected from the group consisting of homopolymers or copolymers of polystyrene, poly(methyl methacrylate), polyacrylamide, poly(ethylene glycol), poly(hydroxyethylmethacrylate), poly(vinyltoluene), or poly(divinylbenzene), and combinations thereof
at least one detectable label selected from the group consisting of a metallic particle, a fluorescent dye, a quantum dot, a quantum barcode, a radiographic contrast agent, or a magnetic resonance imaging contrast agent, and combinations thereof;
chemical functional groups displayed on each of the nanoparticles for attachment of a ligand thereto; and
receptor-specific ligands coupled to the nanoparticle via the chemical functional groups displayed on the nanoparticle, wherein the receptor-specific ligands preferentially associates with said cancerous gastric tissue.

23. A nanotracer as recited in claim 22, wherein the nanoparticle further includes a metallic particle.

24. A nanotracer as recited in claim 22, wherein the receptor-specific ligand is a nutritive substance is selected from the group consisting of sugars, vitamins amino acids, or DNA bases, and combinations thereof.

25. A nanotracer as recited in claim 24, wherein the nutritive substance is folic acid or folate.
